(19) United States
(12) Patent Application Publication
(10) Pub. No.: US 2012/0035279 A1
(43) Pub. Date: Feb. 9, 2012

(54) PROTOCOL FOR SCREENING TRAVELERS

(76) Inventor: Jeffrey E. Miller, San Diego, CA (US)

(21) Appl. No.: 13/205,465

(22) Filed: Aug. 8, 2011

Related U.S. Application Data
(60) Provisional application No. 61/371,625, filed on Aug. 6, 2010.

(51) Int. Cl. C12Q 1/70; A61P 31/00; C40B 20/00; C40B 60/12; C12Q 1/68; C12M 1/34

(52) U.S. Cl. 514/789; 435/6.12; 435/287.2; 435/5; 506/2; 506/39

(57) ABSTRACT

Methods for screening a traveler for an evaluation of the traveler's physical condition or health status by obtaining at least one specimen from the traveler and subjecting the specimen to at least one test that is diagnostic for at least one parameter of the physical condition or health status of the traveler prior to or while the traveler undertakes a journey. Thereafter, the results of the diagnostic test are reported prior to or contemporaneous with the arrival of the traveler at the traveler's destination.

### CROSS-REFERENCE TO RELATED APPLICATION

This application claims the benefit of U.S. Provisional Application No. 61/371,625, filed Aug. 6, 2010, the entirety of which is incorporated herein by reference.

### BACKGROUND OF THE INVENTION

1. Field of the Invention

The present invention relates to the monitoring of the health status of travelers for infectious disease control and national security.

2. Description of the Related Art

The present invention is a tool for global infectious disease monitoring, epidemiological surveillance, and national security. An introduction and overview of the field of Infectious Disease and National Security is provided in the RAND National Defense Research Institute's report of the same name, which was prepared in 2005 for the Office of the Secretary of Defense; the Department of Defense Implementation Plan for Pandemic Influenza (2006); and Unprepared/or a Pandemic, a report by Michael T. Osterholm that appeared in Foreign Affairs in March/April 2007.

The emergence of new infectious diseases and reemergence of diseases capable of generating high global mortality rates, coupled with reluctance and delay in notifying the international community of outbreaks of deadly disease—recently displayed by some foreign officials, and the ease and speed of international travel for human and animal travelers, have highlighted the limitations and deepened expert's concerns in our ability to identify, monitor, track, and contain infectious diseases and dangerous pathogens.

Severe acute respiratory syndrome (SARS), certain strains of influenza (such as avian H5N1), and infectious tuberculosis are just a few of the hundreds of infectious disease agents that present a global health threat. The Center for Infectious Disease Research & Policy (CIDRAP) website states that diseases currently classified as “quarantinable” include: cholera, diphtheria, infectious tuberculosis, plague, smallpox, yellow fever, viral hemorrhagic fevers, SARS, and novel or re-emerging influenza strains with pandemic potential. These are among diseases that are likely to either completely circumvent today's surveillance capabilities, or once identified, simply spread at a rate that will overwhelm global resources, preventing epidemiological reporting networks and tracking systems from containing the spread of disease, likely to lead to a pandemic. Accordingly, our current inability to rapidly identify, report, and control infectious disease agents constitutes a threat to our national security.

International preparedness, cooperation, compliance and resource commitment to infectious disease surveillance and rapid international communication will determine the extent of future outbreaks of infectious disease, and if we continue to rely on current international monitoring systems and communication to learn of potential outbreaks, these outbreaks of communicable infectious disease are likely to create havoc. They have the clear potential to strain global and national resources and overwhelm the Department of Homeland Security and in particular the Centers for Disease Control and the Transportation Security Administration. This could precipitate a breakdown in our ability to effectively manage regional healthcare systems, potentially leading to chaos and civil unrest.

A comprehensive study by the Lowy Institute for International Policy recently determined that a relatively minor pandemic would be expected to claim 1.4 million lives and cost over 300 billion dollars in lost economic output in the global economy. A pandemic similar to the 1918-1919 influenza event would claim 142 million people and have an impact of $4.4 trillion in GDP.

In a recent review, Origins of Major Human Infectious Diseases, which appeared in the journal Nature (Vol. 447; 17 May 2007), Wolfe, Dunavan, and Diamond opined the following: “While the principles of pathogen transmission have not changed during the last 11,000 years, changing modem conditions are exposing us to new pathogen reservoirs and new modes of transmission.” The world is now different from ancient Eurasia, in which most people were farmers and herders in close contact with domestic animals, and in which many of our 25 major diseases of evolved.

While anthrax, Bovine spongiform encephalopathy (BSE), Ebola, and Marburg do kill a high fraction of infected victims (100% in the case of BSE), their inefficient transmission assures few infected victims; 100% of a small number is still a small number. Anthrax and BSE are not transmitted from human to human (absent extremely unusual circumstance such as cannibalism or organ transplants, in the case of SSE-type prions); anthrax is treatable by antibiotics; and human-to-human transmission of Ebola and Marburg fades out after a few transfers and is unlikely to occur in the first place outside of specific settings, such as rural hospitals lacking practices and supplies for controlling infections.

For anthrax, Ebola, hantavirus, and Marburg the rapid onset and severity of symptoms have made identification and containment feasible. Similarly, some emergent Stage-2 pathogens, such as West Nile Virus, hantavirus, and Lyme Disease's agent, have aroused more fear than their lack of human-to-human transmission and modest burden of morbidity and mortality seem to us to warrant. But we acknowledge that virulent pathogens currently exacting a low death toll due to inefficient transmission could become dangerous if they evolved new modes of more efficient transmission (e.g., by aerosolized respiratory droplets), as may have almost occurred in 1989 with the Reston subtype of Ebola virus (Jahrling, et al., 1996).

If not anthrax and Marburg, which pathogens do we instead fear? A straightforward prediction, often neglected in our focus on the exotic, is that some new pandemics will emerge from pathogen taxa of which strains have already caused historical pandemics. Prime candidates are two microbes persisting in animal and/or environmental reservoirs capable of generating new strains: influenza virus and, under local conditions of failing hygiene, possibly cholera. We envision these two important diseases of the past again becoming important diseases of the future. Another prime candidate is tuberculosis, of which new strains have arisen through the development of drug resistance, and which is already causing pandemics among human subpopulations with weakened resistance, such as HIV patients.

A further prediction involves emerging pathogens transmitted by routes that render their spread difficult to control. Sexually transmitted diseases (STD's) fall in this category, because it is difficult to persuade us to abstain from or change our sexual behavior. HIV offers a grim warning: despite its swift and huge global impact, the AIDS epidemic would have been much worse if the sexual transmissibility of HIV (actually rather modest (Wawer, et al., 2005) had equaled that of other sexually transmitted agents such as human papillomavirus (HPV). Similarly, it would be difficult to control emerging pathogens transmitted by our pets (which increasingly include many exotic species as well as traditional domesticated breeds), in chains connecting wild or feral animals to outdoor pets to humans (Daszak, Cunningham & Hyatt, 2000). While we have in some cases reluctantly accepted the culling of millions of farmyard chickens and cows as the price of stemming the spread of avian influenza and BSE, it is hard to imagine killing millions of our family pets, even if those animals did offer a likely entry portal for a dangerous pathogen.

Hence monitoring for the emergence of new STD's and pet-associated diseases would both be good investments.

Still other opportunities exist for novel pathogens to emerge from the big and often-discussed changes associated with modem human societies, such as urbanization, global travel and trade, evolution of drug-resistant microbes, climate change, and increasing numbers of elderly, antibiotic-treated, immuno-suppressed people. For instance, the high rate of urbanization in Africa could transform yellow fever, Chikungunya virus disease, and other African arbovirus diseases into urban diseases, as has already happened with dengue hemorrhagic fever. Globalization, by connecting distant places, permits long-distance transfers of microbes and their vectors, as witnessed by recent North American cases of cholera and SARS brought by infected passengers on jet flights from South America and Asia respectively.

Although we do have a vaccine against yellow fever, most of the world's human population remains unvaccinated (e.g., because of the disease's still-unexplained absence from Asia), so that a yellow fever epidemic especially in China or India could be devastating. Global warming is causing tropical birds and arthropods to expand their ranges into temperate zones (Lovejoy and Hannah, 2005). As tropical arboviruses and arthropod vectors join the ranks of those expanding species, we anticipate temperate flare-ups of many diseases now dismissed as “just” tropical.

All these examples illustrate that future disease control efforts must not only continue the traditional approaches to combating on-going pandemics but must also place more emphasis on “disease forecasting,” the early detection of novel pandemics. As summarized in this review, most of our major human pathogens have emerged from animal reservoirs, and some types of animals and modes of transmission have been especially productive of human killers.”

It is instructive and interesting to note that these authors emphasize the importance of initiating new programs and expanding existing monitoring programs to survey persons in distant countries who are in close contact with animal species that might be vectors for new and emerging infectious agents, yet they failed to connect and see the relevance and failed to propose any monitoring program consistent with and as cost-effective as this invention's targeted approach.

SARS is a viral respiratory illness that infected more than 8,000 people worldwide and killed nearly 800 during an outbreak in 2003. It is spread by close person-to-person contact, mainly when an infected person coughs or sneezes. Global health officials believe a flu pandemic, which could kill millions of people worldwide, is overdue. They are keeping close tabs on the H5N1 strain of bird flu that originated in Asia and which they fear could spark the next pandemic.

Researchers state that SARS' long incubation period of 5-12 days would allow only a small proportion of infected people to be detected by a display of symptoms either at their port of embarkation or during transit. On a 10-hour flight from East Asian countries, a maximum of 21 percent of SARS infected people would be expected to demonstrate symptoms in a timely manner, so as to allow a decision to be made as to their status upon arrival.

Flu has a much shorter incubation period than SARS, on the order of 1-2 days, but the number of infected people detected during flight or through airport screening upon arrival would be small, and most would be missed, according to reports. It has been calculated that if a person were infected with flu two days before their flight they would have a 50 percent chance of developing symptoms during a 10-hour flight. But as most flights are less than 10 hours, it is estimated that less than 10 percent of people would develop symptoms during a flight. Flu sufferers can pass on the illness one or two days before they develop symptoms but it is not clear if the same is true for people infected with SARS.

Screening passengers at the arrival airport is unlikely to prevent the importation of SARS or influenza by infected passengers, raising the possibility that entry screening in a pandemic may not be useful, according to the British Medical Journal (BMJ). A study published by BMJ online looked at the incubation periods for influenza and SARS then estimated the proportion of asymptomatic but infected passengers who would develop symptoms during a flight to the UK. It found the incubation period for SARS was too long to allow more than a small proportion of infected passengers to start showing symptoms during a flight from any destination. The mean percentage of passengers arriving in the UK showing symptoms on arrival was 0.3% for European flights and a maximum of 21% for the longest flights from East Asia. The authors concluded that a larger proportion of people infected with influenza would develop symptoms during the flight, due to the shorter incubation period, but the average was still less than 10%.

In an example of domestic screening, the officer-in-charge for the U.S. Public Health Service Division of Quarantine at Seattle-Tacoma International Airport receives less than two calls a week from an airliner captain reporting that a passenger is ill with symptoms of a possibly quarantinable disease. A quarantine officer meets the plane at the gate and, while other passengers are held on board, examines the ill person and takes a detailed history of symptoms and recent travel.

Foreign quarantine regulations define a passenger as ill if the person has (1) a temperature of 100° F. or greater for two or more days or if the fever is accompanied by a rash, jaundice, or glandular swelling, or (2) diarrhea severe enough to interfere with normal activity. The quarantine inspector has the power to detain persons suspected of having cholera, diphtheria, infectious tuberculosis, plague, suspected smallpox, yellow fever, or suspected viral hemorrhagic fevers such as Lassa and Ebola. In difficult cases, the inspector consults with a contract physician or medical officer with the Centers for Disease Control and Prevention (CDC), National Center for Infectious Diseases. Under a revised protocol effective May 1, 1996, and continuing the Seattle example, the quarantine officer of the Seattle Quarantine Station consults with the CDC and the contract physician if a passenger arrives with suspected viral hemorrhagic fever. With their concurrence, the passenger will be transported under strict isolation to the University of Washington Medical Center. Those with symptoms of other quarantinable diseases will be transported to Harborview Medical Center. The quarantine officer then notifies the state epidemiologist and the Seattle-King County Department of Health. Other passengers who had non-casual contact with the ill person will complete a surveillance form, and all passengers receive a notice to monitor specific illness criteria. If circumstances warrant, close contacts may be urged to obtain prophylactic treatment.

If the cause of illness seems to be food poisoning or a medical condition such as diabetes mellitus, the other passengers and crew are allowed to deplane and the ill person may be released, referred to a physician, or transported to Highline Hospital near the SeaTac Airport. Passengers with symptoms of infectious illnesses such as measles or hepatitis will be referred to physician, with notification given to the appropriate units of the local health department and the CDC for follow-up. In the case of measles, the passengers and crew receive alert notices.

The quarantine inspector completes the initial on-board screening as quickly as possible, usually within five minutes, because “gate time” is expensive to the airlines and the demand for it is intense at this busy airport. Also, passengers tend to be impatient. If the quarantine inspector is paged when the Quarantine Office is closed, however, passengers are held until the inspector or emergency medical technicians on contract to the Public Health Service (PHS) can reach the airport and perform the screening.

Medical inspection of arriving aliens is another major responsibility. All immigrants, refugees, and fiancés/fiancées of U.S. citizens and their minor children are required to have a medical examination overseas. Immigration inspectors at the main port and sub-ports advise the quarantine officer when an immigrant arrives with incomplete or no medical documents. The documents of those who have medical conditions are mailed to the Seattle Quarantine Station for notification. All aliens, including those not routinely required to have a medical examination, may be denied entrance if they exhibit symptoms of a physical or mental disorder that may be excludable under immigration law.

All immigrants who arrive with “Class A” conditions such as tuberculosis (TB), Hansen's disease, or infection with HIV, require a waiver with the name and address of the health provider. Those with TB and Hansen's must have completed treatment. Incomplete treatment for syphilis is a “Class B” condition. A class AB stamp usually is imprinted on the visa. The quarantine officer sends out notification to the state and local health department to follow up. Copies of health papers for immigrants with medical conditions and for all refugees are sent to local health departments and to CDC headquarters.

Additional airport duties include clearance of animals and etiological agents for import. Dogs three months and older must have current rabies vaccination (unless arriving from a rabies-free country) and all cats and dogs are visually inspected. Nonhuman primates may be imported only for scientific, educational, or zoological exhibition purposes, and may be brought in only by registered importers. Special CDC permits are required for some species. For example, primates shipped as pets are seized, re-exported to the country of origin, donated to CDC-approved facilities, or destroyed. Shipments of aquatic fish and plants are inspected for snails, which may carry schistosomiasis, and turtles, which are frequently infected with Salmonella. Certain animal products, for example Haitian goatskins, which may carry anthrax, are banned from import. Etiological agents require a CDC import permit.

The responsibilities of the PHS Quarantine Station at Sea-Tac Airport range far beyond the airport—and beyond the state. The station monitors activity at all international airports and seaports in Alaska, Oregon, and Washington, and the U.S.-Canadian border crossings from Washington to North Dakota. This enormous task is accomplished through cooperation with immigration and customs officers in those locations and through contracting with local physicians for inspection services as needed. All incidents are reported to the Seattle Quarantine Station.

As with airline captains, ship captains also are required to notify the station of a death or illness on board. In one case, a cargo ship left a Mediterranean port, put an ill crewmember ashore in Fort Lauderdale, Fla., and then headed for Astoria, Ore. Florida health authorities notified the Miami Quarantine Station, which notified the Seattle station that the ill crewmember had been diagnosed with active TB. The Seattle station then alerted the Oregon Health Department, TB division, so that every member of the crew who had been in contact with the ill crewmember could be followed up for testing on arrival in Astoria.

Seven to 12 international flights from Asia, Russia, Europe, Mexico, and 30 flights from Canada arrive daily at Sea-Tac. Up to 30 flights a month may arrive with 30 to 50 refugees each. According to the local health department, about 5% of immigrants from some Asian countries have infectious TB, even though their medical documents reported noninfectious status. The introduction of Ebola virus through travelers appears unlikely, but plague or dengue could enter via the shipping industry.

The Seattle Quarantine Station performs a critical function in preventing disease introduction into the Northwest, but it might be likened to a giant sieve. The surveillance and quarantine protocols enforced by its vigilant though overextended staff will catch the more obvious problems. Infected but apparently asymptomatic travelers and aliens will flow through and disperse into the region. The lesson, again, is that our best protection is a strong local public health infrastructure. Public health professionals must be alert for potential disease introduction and prepared to take action to protect the community.

However, there are obvious drawbacks to the present approaches: They are expensive, time consuming, inconvenient for other travelers, suffer from real world difficulties, and, most importantly, will not detect the majority of infectious disease agents in the time necessary to prevent spread of the infectious agents and protect an untainted population at its borders.

### INFLUENZA A—Orthomyxoviridae: Influenzavirus A

Waterfowl and shorebirds, including ducks, are the primary nonhuman reservoirs for influenza A strains. Influenza viruses may be transmitted directly from them to humans (Subbarao, K. & Shaw, 2000), via poultry that have had contact with other birds (Subbarao, K. & Shaw, 2000), (Subbarao, K. & Shaw, 2000)), or by reassortment with avian, porcine, and/or human strains (Hay, A. .J., Gregory, Y., Douglas, A. R. & Lin, 2001).

There are also several concerns that go to gaps in our understanding of disease transmission that impacts our ability to formulate a coherent policy to prevent the transmission of infectious disease. For example, evidence suggests that both pre-symptomatic and asymptomatic individuals transmit influenza virus. Adults shed influenza virus that can be identified by testing at least 1-2 days before the onset of symptoms using current technology, and at least 8% of children infected with influenza A were determined to have been infected by presymptomatic shedding, with virus detected up to six days before the onset of symptoms (Frank AL, et al., Patterns of Shedding of Myxoviruses and Paramyxoviruses in Children, J. Infect Dis. 1981; 144:433-41).

Therefore, when pre-symptomatic or asymptomatic passengers embark on a long trans-Pacific or trans-Atlantic flight, if biological samples are taken just prior to their departure and tested while they are in transit, it is clear that many of these infected passengers are likely to test positive for the infectious agent, even though they may arrive at their destinations displaying no overt symptoms of infection.

For the influenza virus, three conditions need to be met for a new pandemic to start:

1) a novel or long dormant influenza virus hemagglutinin (HA) subtype emerges (or re-emerges); and, the virus infects and replicates efficiently in humans; and

2) the virus spreads easily among humans, and infection cycles in humans are sustained.

It has recently been demonstrated in laboratory studies that the replacement of two amino acids in the receptor binding site of the HA protein of the Asian lineage HPAIV H5NI (Q226L and G228S) improves binding to human receptors of the type similar to those of other human adapted influenza A viruses (Harvey 2004). Gambaryan et al. (2006) have already identified two human isolates from a father and his son infected with H5NI in Hong Kong in 2003, which, in contrast to all other H5NI isolates from humans and birds, showed a higher affinity for these receptors due to a unique S227N mutation at the HA1 receptor binding site. These data are of concern as this is the exact region of the protein identified in the in vitro laboratory studies as critical in changing the tropism of the virus and allowing it to infect humans.

Recently, an infectious disease outbreak reminiscent of the early stages of SARS and bird flu was reported by NEWSWEEK's Barrett Sheridan and various other news services. Pigs are apparently growing sick and dying across China's southeastern Guangdong province. Approximately 3,000 pigs have been infected on hundreds of family farms and about 300 have died. Early reports from Chinese scientists attribute the outbreak to porcine respiratory and reproductive syndrome (PRRS), which first appeared 18 years ago and was originally called Mystery Swine Disease. But certain symptoms of the current outbreak, including massive hemorrhaging, are not consistent with PRRS, and might indicate that the disease, that is most likely caused by a virus, has mutated. The outbreak has renewed fears that a viral pandemic is in the making in southern China.

Again, present methods of screening and surveillance are readily acknowledged, by all experts, to be inadequate to identify and contain potentially deadly infectious disease outbreaks.

During the last outbreak of the SARS virus, Singapore instituted thermal imaging scans to screen all passengers departing Singapore from Singapore Changi Airport. It also stepped up screening of travelers at its Woodlands and Tuas checkpoints with Malaysia. Taipei Taoyuan International Airport SARS Checkpoint with an infrared screening system similar to Singapore's Changi Airport.

Singapore had previously implemented this screening method for incoming passengers from other SARS affected areas but will move to include all travelers into and out of Singapore. In addition, students (and some teachers) in Singapore were issued with free personal oral digital thermometers. Students took their temperatures daily; usually two or three times a day, but these temperature-taking exercises were suspended after this latest outbreak.

Rapid, sensitive, and specific molecular diagnostic screening and surveillance technologies are available that can identify travelers who are infected before they bring potentially deadly infectious disease into our country. Prior to the current invention, we have lacked a mechanism for utilizing these resources to screen travelers for lethal and pandemic infectious diseases.

### SUMMARY OF THE INVENTION

The present invention provides methods and protocols for screening travelers, such as international travelers.

In one aspect, the invention provides a method for monitoring the physical condition or health status of a traveler. This method comprises selecting at least one traveler for an evaluation of the traveler's physical condition or health status, obtaining at least one specimen from the traveler and subjecting the specimen to at least one test that is diagnostic for at least one parameter of the physical condition or health status of the traveler prior to or while the traveler undertakes a journey. Thereafter, the results of the diagnostic test are reported prior to or contemporaneous with the arrival of the traveler at the traveler's destination.

A further aspect of the invention provides a method for verifying the physical condition or health status of a traveler comprising: obtaining the results of at least one test that is diagnostic for at least one parameter of the physical condition or health status of a traveler previously selected for an evaluation, which test was conducted prior to or while the traveler undertook a journey and said results are obtained prior to or contemporaneous with the arrival of the traveler at the traveler's destination; and determining the status of and/or further activities involving the traveler based upon the test results.

A further embodiment comprises a preconfigured unit that can be deployed in a timely way in proximity to or within a travel embarkation setting that contains testing supplies and or materials that allow practice of a method for verifying the physical condition or health status of a traveler by obtaining the results of at least one test that identifies the presence of at least one infectious agent in a traveler previously selected for an evaluation. In one embodiment, the preconfigured unit comprises a vehicle, such as a van, recreational vehicle, or tractor trailer containing testing supplies and or materials that allow practice of a method for verifying the physical condition or health status of a traveler. In another embodiment, the preconfigured unit comprises a temporary or semi-permanent structure that may be quickly assembled and disassembled. In some embodiments the unit may be fully assembled in less than one hour. In some embodiments, the preconfigured unit is a collapsible unit. In some embodiments, the preconfigured unit is provided as a self-contained unit in a shipping container or on a pallet. Such a self-contained unit may be configured so as to be compatible with universal-sized shipping containers or with transport by cargo jet. Such a self-contained unit may also be configured so that it is easily stored on the site of or adjacent to a point of transit such as a terminal, depot, port, or station. In some embodiments, the preconfigured unit contains an independent power supply, such as solar cells, batteries or generators. In addition to sample collection and sample storage materials, the preconfigured unit could also advantageously comprise one or more of the following: consent forms; signage; blood collection equipment; packaging for transporting a sample; electronic communication equipment; bar code or 2D code printer or reader; boarding pass or passport scanner; sample labeling equipment; thermal human scanning equipment; human temperature measurement equipment; digital photography or video equipment; sample testing equipment to perform a diagnostic assay on a sample taken from a traveler

### DETAILED DESCRIPTION OF THE PREFERRED EMBODIMENT

In accordance with the present invention, a method for monitoring the physical condition or health status of a traveler is provided. The method comprises selecting at least one traveler for an evaluation of the traveler's physical condition or health status, obtaining at least one specimen from the traveler and subjecting the specimen to at least one test that is diagnostic for at least one parameter of the physical condition or health status of the traveler prior to or while the traveler undertakes a journey. Thereafter, the results of the diagnostic test are reported prior to or contemporaneous with the arrival of the traveler at the traveler's destination.

A further aspect of the invention provides a method for verifying the physical condition or health status of a traveler comprising: obtaining the results of at least one test that is diagnostic for at least one parameter of the physical condition or health status of a traveler previously selected for an evaluation, which test was conducted prior to or while the traveler undertook a journey and said results are obtained prior to or contemporaneous with the arrival of the traveler at the traveler's destination; and determining the status of and/or further activities involving the traveler based upon the test results.

A further aspect of the invention provides a method for determining the status of and/or further activities involving the traveler based upon the test results contained in said report. Further activities may comprise questioning the traveler, verifying test results, performing additional tests, isolating, quarantining, treating, and/or refusing or restricting further travel.

This invention provides, for the first time, a method by which the physical condition or health status of a traveler is ascertained or determined concomitant with or after the initiation of travel, results are confirmed, and finalized confirmed test results are reported to authorized agents at the predetermined destination prior to, or contemporaneously with the traveler's arrival. Testing, confirmation, reporting, and triage occur in real-time, and test results may be generated and confirmed prior to the onset of any symptoms.

In accordance with the present invention, a method for rapidly identifying asymptomatic infected individuals is disclosed. Using the present invention, identification of the infectious disease or infectious agent is accomplished, and verified, without delaying travel plans. In certain embodiments of this invention authorities may even be provided with detailed information on the type, strain or Glade of the infectious agent. Information obtained on individuals so identified can be used and applied according to the individual laws governing the countries affected. Passengers' samples can be coded to maintain some level of anonymity, but obviously it is desirable that individual travelers may be identified, if the results of the screening warrant further action that can be limited to such individuals.

Unless otherwise defined, all technical and scientific terms will be used in accordance with the common understanding of persons ordinarily skilled in the art to which the present invention is related. As used herein, the following terms shall have the assigned meanings unless a contrary definition is clearly indicated from the context in which the term is used.

The term test result indicates a positive; a negative or an indeterminate finding generated from a traveler's sample as determined following the interpretative criteria from a validated test or assay.

The present method for determining the health status of a traveler can occur during the latency or incubation period of many infectious diseases prior to the onset of any overt symptoms.

The present method for determining the health status of a traveler can also be useful for travelers who are infected but asymptomatic and never display any overt symptoms.

### Selection of Travelers to be Evaluated

Prior to the time a traveler or a cohort of travelers (human, animal, and the like) board an airplane, ship, train, or other conveyance, a determination will be made as to which travelers are to be evaluated (or screened). This allows a variety of selection criteria to be employed, depending on the seriousness and known progression patterns of the disease for which testing is performed. Although employing the present invention with a highly specific selection criterion (example: select for testing all passengers that indicate that they may have been exposed to pandemic infected individuals) will derive some benefit, it is clear that the maximum benefit will be obtained if a broad set of travelers in a particular cohort or class (example: select for testing all passengers coming from a pandemic area) is selected for screening. This latter strategy would enable more asymptomatic travelers to be detected and avoid the inherent bias towards suspected symptomatic travelers, who would likely already be detected by more traditional means. In addition, broad selection criteria (up to and including all international travelers, in case of extreme health emergency) may be more desirable socially or politically to avoid the stigma of selective screening, and potential claims of discrimination.

### Specimen Collection

There are numerous means of collecting specimens, typically but not exclusively biological specimens, currently known and in use, and many more techniques will be developed. Common techniques and specimens include sampling of hair, skin (typically sloughed skin cells), and other bodily tissues, blood, saliva, sputum, urine, and other bodily fluids, and physical emissions such as exhalants (containing chemicals or biological agents associated with the subject) are sampled for various purposes.

Any means of specimen collection, and any appropriate type of specimen, previously known or later developed, would be expected to find use in the present invention. Clearly, however, relatively rapid and non-invasive techniques are preferred, such as saliva, mouth, throat, and nasal swabs, and/or exhalants and the like, both to increase the efficiency of the screening, and to reduce the instances of subject noncompliance due to discomfort or perceived inconvenience. For example, exhalant specimens could be obtainer by a sampling device or “sniffer” that monitors individual travelers or other items as they pass through a screening area (e.g., an X-ray booth), and the results could be correlated to the sampled entity by some form of coding (e.g., a bar code or RFID chip included in a boarding pass or other item). This would allow broad range sampling with minimal inconvenience to the travelers.

### Diagnostic Screening

The present invention can be applied in the detection of virtually any physical condition that is deemed desirable to detect in travelers and upon which to base a determination of their status and further activities, treatment, and/or travel destination (such as onward toward their intended destination, a quarantine facility, or a return to their place of embarkation).

In order for the present invention to be implemented most effectively, it is desirable to employ rapid, sensitive, and specific diagnostic procedures, as the delay times between initial screening and arrival of the traveler would typically range from a few hours to 36 hours (for complicated international air-travel itineraries), or longer for certain modes of travel (e.g., ships or trains).

A series of available technologies fit the performance criteria for sensitivity and specificity required for this invention. First among these are amplification technologies such as polymerase chain reaction (PCR), and, when required because the initial target is RNA, reverse transcriptase polymerase chain reaction (RT-PCR). These highly specific and sensitive technologies are readily available, the technologies can generate valid test results with almost any specimen that contains nucleic acid, the instrumentation is inexpensive, and persons of moderate technical ability can be easily trained to generate reproducible and valid test results. Further, these technologies can generate an initial test result within several hours of sample procurement, and the test result can be verified with a second test result prior to the arrival of the passenger. Isothermal strand displacement amplification (SDA), ligase chain reaction (LCR), loop-mediated isothermal amplification (LAMP), as well as signal amplification technologies such as hybrid capture may also be useful and may have specific advantages in certain situations.

A recently developed technique is the bio-aerosol mass spectrometry (BAMS) system, the only known instrument that can detect and identify spores at low concentrations in less than 1 minute. BAMS can successfully distinguish between two related but different spore species. It can also sort out a single spore from thousands of other particles—biological and nonbiological—with no false positives. Livermore's Laboratory Directed Research and Development (LDRD) Program funded the biomedical aspects of the BAMS project, and the Department of Defense's Technical Support Working Group and Defense Advanced Research Project Agency funded the bio-defense efforts.

BAMS may prove useful to instantly identify and exclude boarding by travelers afflicted with certain infections or diseases if sufficient hardware and personnel allow deployment at airport departure area entry points. But it may also prove useful in identifying infections or diseases in conjunction with analysis of collected specimens at a departure airport lab facility, requiring the use of passenger transit time and implicating the present invention. Similarly, it may prove useful for some diseases, infections and conditions in conjunction with parallel screening with alternate technologies for which passenger transit time is needed, again implicating the current invention.

As an alternative for rapid screening, researchers at MIT's Lincoln Laboratory and Department of Biology report in the Jul. 11, 2005, issue of Science that they have created a fast, powerful new sensor that can identify everything from SARS to bioterrorist agents. Called CANARY (Cellular Analysis and Notification of Antigen Risks and Yields), the sensor gives cells that comprise the body's first line of defense against viruses and bacteria the ability to glow like jellyfish in the presence of contaminants.

CANARY has potential applications in medical diagnosis (it can detect pathogenic bacteria, viruses, fungi, protozoa and proteins from samples in a variety of formats); environmental uses (it can test water quality, indoor air quality for diseases such as Legionnaire's, food safety and agricultural pathogens such as foot-and-mouth); and defense (it can detect anthrax and other bio-warfare agents). While existing sensors are based on chemical reactions that can take several hours to work and require several thousands of particles to detect, CANARY can detect as few as 50 colony-forming units of the plague bacterium in less than three minutes.

Again, and depending on the disease and technology involved and the deployment of equipment and personnel, CANARY may ultimately prove useful to almost instantly identify and exclude boarding by travelers afflicted with certain infections or diseases. But it may also prove useful in identifying infections or diseases in conjunction with parallel screening with alternate technologies and/or with specimen transport and analysis for which air transit time is needed, implicating the current invention.

Especially in the case of bio-terrorism, Rider said, “It's desirable to have something faster and more sensitive.” Rider created a special strain of mouse B cells and identified a jellyfish gene that allows the organism to produce a protein that glows. In a collaboration with Jianzhu Chen, associate professor of biology in the MIT Center for Cancer Research who studies the molecular mechanisms underlying the development and function of the immune system, Rider then genetically engineered B cell lines that glowed in response to specific bacteria and viruses, allowing extremely rapid identification of disease once the specimen is in the laboratory.

In addition to creating cell lines to detect a variety of specific pathogens, including anthrax, plague, smallpox, equine encephalitis and Chlamydia, a sexually transmitted disease, the Lincoln Lab engineers developed hardware to house the cells. The cells are placed in a luminometer, a container that keeps the cells alive in a test tube and monitors their luminescence in the presence of pathogens. A laptop computer provides readout of the cells' response; these results could them be emailed to appropriate authorities, including the Centers for Disease Control, the Transportation Security Administration, and in case of urgent infection indicative of a pandemic risk, Federal and State quarantine officials in the arrival airport.

However, there are numerous means of rapidly detecting infectious agents or other diagnostic indicia in biological specimens that are currently known and in use, and many more techniques such techniques will be developed in the future. Depending upon the amount of time available for the screening procedure, these and other techniques of greater or lesser rapidity can be employed in the present invention.

### Reporting Results and Further Determination/Action

Once the results of the diagnostic screening(s) is obtained, it can readily be reported to the appropriate entity, for example to the carrier or organization tasked and responsible for operation of the vehicle; a regional center of the Centers for Disease Control; or to a person or entity of authority at the traveler's destination; most commonly a facility at the airport, ship terminal, or bus or train station, for a determination of the traveler's status and any further appropriate action. As a result of this screening report, it is anticipated that the large majority of the cohort of travelers would be found free of any detectable physical condition that might inhibit their travel plans, and would be allowed to continue towards their ultimate destinations.

Other travelers, who have been determined to possess a physical condition of interest, would then be handled appropriately based upon their determined status. Depending upon the physical condition detected, current further actions typically include further testing or second-site confirmation of a test result (using PCR this likely can occur prior to arrival), quarantine, referral to a healthcare provider for treatment, or a mandatory return to their place of embarkation.

This invention successfully addresses one of the most difficult tasks facing the public health service: Reliably identifying, sequestering, and tracking infected asymptomatic individuals arriving at a debarkation site from other locations to prevent spread of infectious disease in the arrival location. Authorities will then be in a position to respond according to applicable laws governing the testing of individuals and control of infectious agents.

Because direct amplification or immunoassay based testing of material collected from nasal or throat swabs is more likely to identify presence of infectious disease agents prior to the onset of symptoms, fewer travelers harboring and infected with pathogens are likely to gain entry into the country of destination without an opportunity to intervene and take appropriate action.

In addition to public health benefits, the invention (by expanding options to more effectively limit the spread of a pandemic based on identification and if necessary quarantine of infected individuals) has potentially strong Constitutional and international treaty compliance advantages.

Plainly, holding passengers because a Transportation Safety Administration (TSA) employee with little or no medical training thinks they might be sick based on visible inspection is ripe with risk of risk of discriminatory enforcement, invasion of privacy and other Constitutional risks. See, e.g., comments of the American Civil Liberties Union on the Oct. 19, 2005, Memorandum of Understanding (MOU) between the Department of Homeland Security and the Center for Disease Control. Cite: http ://www.aclu.org/privacy/spying/25332res20060425.html (with attached link to copy of MOU text). This issue is of particularly Constitutional concern given the comparative ineffectiveness of a strategy of holding people based on visible symptoms—an approach that will lead to both under-identification (due to the time lapse during incubation, in other words after infection and before presentation of symptoms) and over-identification of allegedly ill persons; many pandemic flu disease symptoms will obviously mimic those of ordinary cold and flu infections.

Here, and depending on the individual disease involved, it may be practical and most effective to identify, warn and if necessary quarantine individuals testing positive for actual infection, rather than simply holding travelers exhibiting visible symptoms that might indicate actual infection. Such an approach may also assist the United States with compliance with international treaty obligations. For example, the MOU between the Department of Homeland Security and the Center for Disease Control cited above has been criticized as implying that airline passenger travel and identity information that is obtained from European countries will be used for other than the agreed upon limited purpose of detection of serious criminals. Cite: http://www.cidrap.umn.edu/cidrap/content/bt/bioprep/news/apr2706data.html [analysis of Center for Infectious Disease Research & Policy Academic Health Center—University of Minnesota]. Use of such information to broadly define who has traveled to a pandemic area may become of less importance or entirely unnecessary if a screening program based on testing while the passenger is in flight it adopted.

Without taking positions as to the complex Constitutional positions raised, it is plain that privacy rights will be served by eliminating or lowering dependence upon “visible inspection” type screening systems. The importance of protecting the privacy rights has been acknowledged at least implicitly by the efforts of even proponents of strong homeland security measures to ensure that information is competently collected and used only for the purposes intended. See Oct. 19, 2005, Memorandum of Understanding, cited above (passim, and Appendix specifying limits on information use). At the other end of the spectrum, see Constitutional criticism submitted to and republished by the Center for Disease Control of a potential failure to apply “21st Century Medicine” in infectious disease screening: http://www.cdc.gov/Ncidod/dq/nprm/comments/2006Mar1_ACLU. pdf

### EXPERIMENTAL

In the experimental disclosure which follows, the following abbreviations apply: eq (equivalents); M (Molar); mM (millimolar); μM (micromolar); N (Normal); mol (moles); mmol (millimoles); μmol (micromoles); nmol (nanomoles); kg (kilograms); gm (grams); mg (milligrams); μg (micrograms); ng (nanograms); L (liters); mL (milliliters); μL (microliters); cm (centimeters); mm (millimeters); μm (micrometers); nm (nanometers); V (volts); μF (microfarads) and ° C. (degrees Centigrade).

In the following Examples, unless otherwise specified, oligonucleotides are obtained from IDT, or can be synthesized, e.g., on an Applied Biosystems DNA synthesizer according to the manufacturer's instructions. Thermus aquaticus DNA polymerase I is obtained from Perkin-Elmer Cetus. All standard molecular biology techniques are performed according to Sambrook et al. (1989) or Berger and Kimmel (1987), herein incorporated by reference. Nucleic acid sequences disclosed herein are divided into 10-mer or smaller oligonucleotides as a matter of convenience, and should be interpreted as continuous sequences unless otherwise indicated.

Laboratories are desirably compliant with the CDC directive that all laboratories analyzing or referring specimens or samples that may contain microbial agents or biological toxins function as sentinels in the nation's Laboratory Response Network (LRN). All tests are validated tests, whether commercial, or homebrewed assays. When sentinel clinical laboratories are unable to rule out possible bioterrorism agents using standard LRN tests, they refer suspicious isolates or specimens to their collaborating LRN reference laboratory in San Diego, California. At the LRN reference laboratory, additional analyses are conducted using standardized, validated confirmatory assays made available through the CDC.

### Direct Detection Methods

Direct detection methods do not produce an isolate and may be inadequate for surveillance or definitive characterization of pandemic strains; especially if these studies require cell culture or other technologies requiring growth or replication of viable isolates. Nevertheless, owing to their relatively rapid turnaround time, safety, and stability, direct detection methods play an important role in identifying infectious diseases with pandemic potential.

The following are a series of tests and technologies listed and available through the Centers for Disease Control for detection of Influenza:

### Reverse-Transcriptase, Polymerase Chain Reaction (RT-PCR) Assays

RT-PCR assays target conserved genes, such as genes for the matrix (M) protein, for genus-level identification. Hemagglutinin and neuraminidase targets are used for specific identification of avian subtypes.

When compared with cell culture the sensitivity of RT-PCR has been reported to be in the range of 90% to 100%. However, several researchers have reported significantly higher clinical sensitivity numbers with RT-PCR, possibly reflecting its ability to detect nonviable virions (Coiras 2003, Hayden 2002, Herrmann 2001, Pachucki 2004, Wallace 1999).

In February 2006, the Food and Drug Administration (FDA) announced clearance of an Influenza A/H5 (Asian Lineage) Virus Real-Time Reverse Transcription-Polymerase Chain Reaction (RT-PCR) Primer and Probe Set and inactivated virus as a source of positive RNA control for the in vitro detection of highly pathogenic influenza A/H5 virus (Asian lineage) (CDC 2006: New laboratory assay for diagnostic testing of avian influenza A/H5 [Asian lineage]). These reagents and assay protocols were distributed by CDC to state and city LRN (Laboratory Response Network) laboratories. Testing with the new assay is limited to LRN-designated laboratories.

Multiplex real-time RT-PCR assays have been developed for specific detection of H5N1 (See References: Kessler 2004, Ng 2005, Payungporn 2005).

While culture of specimens from possible avian influenza (H5N1) cases is not recommended without strict containment and specific registration, RT-PCR can be conducted using BSL-2 facilities and practices (HHS 2005: Pandemic influenza plan).

Samples positive by RT-PCR for a novel influenza subtype should be forwarded to a public health laboratory (if testing was conducted at a private laboratory) or to CDC for confirmation (see References: HHS 2005: Pandemic influenza plan).

### Molecular Microarray Tests Using Flow-Through Chip Technology

A molecular microarray for influenza typing and subtyping using a flow-thru chip platform was initially described in 2004 (see References: Kessler 2004), and two reports released in August 2006 involved a study of the FluChip-55 diagnostic microarray and showed that the test could be a valuable tool in identifying influenza viruses (see References: Mehlmann 2006, Townsend 2006). The FluChip used in the study contained 55 sequences of RNA representing a variety of type A and type B flu viruses, including H3N2, H1N1, and H5N1. Combined results after two rounds of testing showed that the FluChip allowed users to obtain correct information about both type and subtype from 72% of 72 samples tested. Full information on type, but only partial information on subtype, was obtained for an additional 13% of the samples, while 10% of the samples could be identified by type only (no information about subtype). The entire analysis time was less than 12 hours.

Scientists recently have developed an improved microarray test referred to as the “MChip,” which has several advantages over the FluChip. While the FluChip is based on three influenza genes-hemagglutinin (HA), neuraminidase (NA) and matrix (M)-the MChip is based on only the M gene segment, which mutates much less rapidly. A recent evaluation demonstrated that the assay exhibited a clinical sensitivity of 97% and clinical specificity of 100% (see Nov. 15, 2006, CIDRAP News Story).

Nucleic acids can be amplified by a modification of the technique referred to as the loop-mediated isothermal amplification (LAMP) method, and the corresponding diagnostic test for SARS is described in detail in U.S. patent application No. 20070099178. Because the SARS coronavirus is an positive strand RNA virus, the corresponding amplification test used for diagnosis requires inclusion of a reverse transcriptase enzyme and is therefore designated an RT-LAMP test.

SARS genome patent; Haagmans; Bartholomeus Leonardus; et al.: U.S. patent application 20070053878

### RT-PCR

A one-step RT-PCR was performed in 50 μL reactions containing 50 mM Tris.HCl pH 8.5, 50 mM NaCl, 4 mM MgCl₂, 2 mM dithiotreitol, 200 μM each dNTP, 10 units recombinant RNAsin (Promega, Leiden, the Netherlands), 10 units AMV RT (Promega, Leiden, The Netherlands), 5 units Amplitaq Gold DNA polymerase (PE Biosystems, Nieuwerkerk aan de Ijssel, The Netherlands) and 5 μL RNA. Cycling conditions were 45 min. at 42° C. and 7 min. at 95° C. once, 1 min at 95° C., 2 min. at 42° C. and 3 min. at 72° C. repeated 40 times and 10 min. at 72° C. once.

### Primers Used for Diagnostic PCR

For the amplification of the SARS virus' genetic material, specially designed primers were employed, as noted in the source. These primers amplify a 149 by fragment of the polymerase gene.

### Rapid Testing to Identify Influenza A/H5 (Asian lineage) Virus

Influenza A/H5 (Asian lineage) Virus Real-time RT-PCR Primer and Probe Set was used to screen samples. The test provides preliminary results on suspected H5 influenza samples within four hours once a sample arrives at the lab and testing begins. Previous testing technology would require at least two to three days to render results. If the presence of the H5 strain is identified, then further testing is conducted to identify the specific H5 subtype (e.g., H5N1). Specimens were collected from the upper respiratory tract (consistent with CDC guidelines). Specifically they were taken from posterior-pharyngeal (throat) swabs, which provide the highest viral yield; nasal swabs with nasal secretions (from the anterior turbinate areas), or nasopharyngeal aspirates or swabs (these specimens were determined to be more appropriate for seasonal influenza and may yield lower sample for avian influenza).

The presence of influenza A specific RNA is detected through the reverse transcription-polymerase chain reaction (RT-PCR) that targets fragments of the M gene, the most highly conserved genome segment of influenza viruses (Fouchier 2000, Spackman 2002), or the nucleocapsid gene (Dybkaer 2004). When a positive result is obtained, RT-PCRs amplifying fragments of the haemagglutinin gene of subtypes H5 and H7 are run to detect the presence of notifiable AIVs (Dybkaer 2004, Spackman 2002). When positive again, a molecular diagnosis of the pathotype (LP versus HP) is feasible after sequencing a fragment of the HA gene spanning the endoproteolytic cleavage site. Isolates presenting with multiple basic amino acids are classified as HPAI. PCRs and other DNA techniques are being designed for the detection of Asian lineage H5N1 strains (Collins 2002, Payungporn 2004, Ng 200S). Non-H5/H7 subtypes can be identified by a canonical RT-PCR and subsequent sequence analysis of the HA-2 subunit (Phipps 2004). There are also specific primers for each NA subtype. A full characterization might be achievable within three days, especially when real time PCR techniques are used (Perdue 2003, Lee and Suarez 2004). However, DNA chips are in development that should further streamline the typing of AI viruses (Li 2001, Kessler 2005). An exclusion diagnosis is possible within 4-6 hours.

Preparedness and Communication: A molecular testing laboratory facility that is compliant with international and Centers for Disease Control standards for: facilities; personnel; sample collection, accessioning, storage, extraction, interpretation and reporting. Surveillance and Detection: (tests; instrumentation; validation; sample storage). Response and Containment: (secure communication, results verification).

### U.S. Patent Application 20070092871—Microarray for Influenza and SARS

In the face of concerns over an influenza pandemic, identification of virulent influenza isolates must be obtained quickly for effective responses. Knowledge of the exact strain, origin of the strain, and probable characteristics of the virus are critical for surveillance of a disease outbreak and preventing the spread of the disease. Rapid subtype identification of flu is not always straightforward. Simple serological tests on infected individuals are awkward to administer and are an ineffective tool for monitoring viruses undergoing a high rate of mutation or rapid recombination. RT-PCR assays have better sensitivity but are problematic in scenarios where new strains of virus emerge or mixtures of viruses exist. RNA viruses such as flu undergo antigenic shift and genetic drift as they circulate through populations. Tracking these changes and keeping abreast of evolving viral variants is the key to effective vaccination and can provide insight as to why certain strains of flu are drug resistant or more lethal to infected hosts. In addition, influenza isolates circulating in non-human populations (e.g., birds, pigs, and dogs) must also be monitored on an ongoing worldwide basis to detect virulent isolates that have the potential to infect humans directly or recombine with common human strains of flu to produce lethal hybrids. In many situations, the identification of the circulating subtype (e.g., by simple serotype or a simple RT-PCR test) is not sufficient, and specific knowledge of the genetic makeup of the virus is required. For example, the avian H5N1 virus has significant potential for further recombination with common human strains (e.g., H3N2) or other non-human strains common in avian populations (H7 and H9 strains). The H5N1 subtype is also difficult to identify because of the lack of sensitivity and specificity of many of the commercial tests. In addition, genotype Z, the dominant H5N1 virus genotype circulating in Vietnam and Thailand contains a mutation that is associated with resistance to amantadine and rimantadine. Because of the high susceptibility in humans and resistance to antibiotics of this isolate, neuraminidase inhibitors must be given within 48 hours of onset of illness to be effective. Thus rapid and specific identification of this subtype and accurate sequence information is crucial for proper treatment.

### SARS Paper: http://www.cdc.gov/ncidod/EID/vol10no2/03-0759.htm

Nucleic Acid Extraction. Nucleic acids were recovered from clinical specimens using the automated NucliSens extraction system (bioMérieux), Following manufacturer's instructions, specimens received in NucliSens lysis buffer were incubated at 37° C. for 30 min with intermittent mixing, and 50 μL of silica suspension, provided in the extraction kit, was added and mixed. The contents of the tube were then transferred to a nucleic acid extraction cartridge and processed on an extractor workstation. Approximately 40-50 μL of total nucleic acid eluate was recovered into nuclease-free vials and either tested immediately or stored at −70° C.

### Primers and Probes

Multiple primer and probe sets were designed from the Urbani strain of SARS-CoV polymerase 1b and nucleocapsid gene sequences (15) by using Primer Express software version 1.5 or 2.0.0 (Applied Biosystems) with the following default settings: primer melting temperature (TM) set at 60° C.; probe TM set at 10° C. greater than the primers at approximately 70° C.; and no guanidine residues permitted at the 5′ probe termini. All primers and probes were synthesized by standard phosphoramidite chemistry techniques at the Biotechnology Core Facility at the Centers for Disease Control and Prevention (CDC). TaqMan probes were labeled at the 5′-end with the reporter molecule 6-carboxy-fluorescein (FAM) and at the 3′-end with the quencher Blackhole Quencher 1 (Biosearch Technologies, Inc., Novato, Calif.). Optimal primer and probe concentrations were determined by cross-titration of serial twofold dilutions of each primer against a constant amount of purified SARS-CoV RNA. Primer and probe concentrations that gave the highest amplification efficiencies in this study were selected for further study (Table I).

### Real-Time RT-PCR Assay

The real-time RT-PCR assay was performed by using the Real-Time One-Step RT-PCR Master Mix (Applied Biosystems). Each 25 μL reaction mixture contained 12.5 μL of 2X Master Mix, 0.625 μL of the 40X MultiScribe and RNase Inhibitor mix, 0.25 μL of 10 μM probe, 0.25 μL each of 50 μM forward and reverse primers, 6.125 μl, of nuclease-free water, and 5 μL of nucleic acid extract. Amplification was carried out in 96-well plates on an iCycler iQ Real-Time Detection System (Bio-Rad, Hercules, Calif.). Thermocycling conditions consisted of 30 min at 48° C. for reverse transcription, 10 min at 95° C. for activation of the AmpliTaq Gold DNA polymerase, and 45 cycles of 15 sec at 95° C. and 1 min at 60° C. Each run included one SARS-CoV genomic template control and at least two no-template controls for the extraction (to check for contamination during sample processing) and one no-template control for the PCR-amplification step. As a control for PCR inhibitors, and to monitor nucleic acid extraction efficiency, each sample was tested by real-time RT-PCR for the presence of the human ribonuclease (RNase) P gene (GenBank accesssion number NM_006413) by using the selected primers and probes.

The assay reaction was performed identically to that described above except that primer concentrations used were 30 μM each. Fluorescence measurements were taken and the threshold cycle (CT) value for each sample was calculated by determining the point at which fluorescence exceeded a threshold limit set at the mean plus 10 standard deviations above the baseline. A test result was considered positive if two or more of the SARS genomic targets showed positive results (CT<45 cycles) and all positive and negative control reactions gave expected values.

Clinical specimens submitted to CDC for SARS-CoV testing that gave positive results were confirmed with a TaqMan real-time RT-PCR assay based on three different primer and probe sets (Table 1). This assay was performed independently in a separate laboratory using newly extracted nucleic acid from a second specimen aliquot. The confirmatory assay used the SuperScript One-Step RT-PCR (Invitrogen Corp., Carlsbad, Calif.) and the Mx4000 Multiplex Quantitative PCR system (Stratagene, La Jolla, Calif.).

### Synthesis of RNA Transcripts

Template for the nucleocapsid gene RNA was plasmid DNA (pCRII, Invitrogen Corp.) containing a full-length copy of the open reading frame for the SARS-CoV nucleocapsid gene oriented behind a T7 promoter. The plasmid was linearized by digestion with Spe I. The template for the polymerase RNA was a RT-PCR product generated by using the selected primers. Approximately 1 mg of RNA from Vero cells infected with SARS-CoV was used in RT-PCR reactions performed by using the SuperScript RT-PCR kit (Invitrogen Corp.) according to the manufacturer's instructions; both templates were purified by phenol-chloroform extraction and ethanol precipitation before being used for in vitro transcription. RNA was synthesized in vitro by using the MegaScript kit (Ambion Inc., Austin, Tex.) according to the standard protocol. Synthetic RNA was treated with RNase-free DNase before being purified by phenol-chloroform extraction and ethanol precipitation. The concentration of RNA was determined by use of UV spectroscopy. Synthetic RNA was positive sense and 1,369 nt in length for N and 325 nt in length for polymerase (Development of a Real-Time Reverse Transcriptase PCR Assay for Type A Influenza Virus and the Avian H5 and H7 Hemagglutinin Subtypes. Spackman, et al., J. Clin. Microbiology, September 2002, p. 3256-3260, Vol. 40, No. 9).

Thus it has been shown that the present invention provides methods and compositions to select at least one traveler for an evaluation of said traveler's physical condition or health status; obtain at least one biological specimen from said traveler; subject said specimen to at least one test that is diagnostic for at least one parameter of the physical condition or health status of the traveler prior to or while the traveler undertakes a journey; and report the results of said diagnostic test prior to or contemporaneous with the arrival of the traveler at the traveler's destination.

It has also been shown that these methods and compositions are sufficiently rapid and sensitive to allow for testing and reporting consistent with this invention.

### Testin of Asymptomatic Individuals

Molecular testing is effective in identifying a disease state in travelers prior to the appearance of identifiable symptoms. For example, at Day 1 of an influenza infection an otherwise healthy individual has a virus shed of 3.0 log₁₀ TCID₅₀, which precedes the onset of symptoms like sore throat, myalgia, headache, cough, and malaise. The Limit of Detection (LoD) for rRT-PCR Panels capable of detecting A/Hawaii/15/2001 (H1N1), A/New Caledonia/20/1999 (H1N1) and A/Wisconson/55/2004 (H3N2), on the other hand, is below 3 TCID₅₀. Thus, molecular testing techniques allows for the identification of travelers infected with the influenza virus even before the traveler is aware that they are ill. Identification and sequestration of these individuals can aid in slowing the spread of infection during an epidemic or pandemic.

The deployable unit contemplated herein will have some or all of the materials needed to rapidly obtain, accession, process, test, interpret, store, and transmit or convey results generated from a traveler's clinical specimen for use in one or more of the methods disclosed herein. Typically, it will include sample collection materials, such as a cheek swab, nasal swab, throat swab, urine collection materials, or blood collection materials, such as a finger stick device or IV needle set with collection tube. Further, if the sample will be tested off-site, the unit or kit may include packaging materials. Equipment for correlating the sample with the traveler who provided it are typically also included, which may include a electrical generator and water supply, so that the unit is not dependant on other infrastructure, bar code, 2D code, RF tag, or other identification equipment, a printer, a boarding pass or passport scanner, photography equipment, fingerprint or other biometric ID equipment, electronic storage media linked to the correlating or ID equipment, and the like. Instruments for reading physical data from the traveler are also contemplated in some embodiments, such as blood pressure instruments, instruments for reading patient temperature (such as contact or non-contact thermometers or scanners), and the like. Thermal scanning instruments for reading the temperature of travelers as they pass by are also known, and can be used to assist in selecting travelers for screening. Preferably, such an instrument will have a video imaging capability for showing both an individual traveler and his or her skin temperature. In some embodiments, the unit includes ancillary material, such as one or more of a table covering, signage identifying the collection unit and/or giving instructions to passengers, consent forms, information forms, and the like. Questionaires to evaluate risk factors and assist in selection of travelers for testing are also contemplated. For some types of assays, it may include portable diagnostic instruments. These may include instruments for one or more of the types of assays described herein, such as portable immunoassay readers, PCR instruments, flow cytometers, and the like.

All patents and patent applications cited in this specification are hereby incorporated by reference as if they had been specifically and individually indicated to be incorporated by reference.

Although the foregoing invention has been described in some detail by way of illustration and Example for purposes of clarity and understanding, it will be apparent to those of ordinary skill in the art in light of the disclosure that certain changes and modifications may be made thereto without departing from the spirit or scope of the appended claims.

### REFERENCES

Subbarao, K. & Shaw, M. W. Molecular aspects of avian influenza (H5N1) viruses isolated from humans. Rev. Med. Virol. 10, 337-348 (2000).

Lin, Y. P. et al. Avian-to-human transmission of H9N2 subtype influenza A viruses: relationship between H9N2 and H5N1 human isolates. Proc. Natl. Acad. Sci. U.S.A. 97, 9654-9658 (2000).

Hay, A. J., Gregory, V., Douglas, A. R. & Lin, Y. P. The evolution of human influenza viruses. Philos. Trans. R. Soc. Lond. B. Biol. Sci. 356, 1861-1870 (2001).

Jahrling, P. B. et al. Experimental infection of cynomolgus macaques with Ebola-Reston filoviruses from the 1989-1990 U.S. epizootic. Arch. Virol. Suppl. 11, 115-134 (1996).

Daszak, P., Cunningham, A. A. & Hyatt, A. D. Emerging infectious diseases of wildlife-threats to biodiversity and human health. Science 287, 443-449 (2000).

Montalto, N., An Office-Based Approach to Influenza: Clinical Diagnosis and Laboratory Testing. Am. Fam. Phys. 67, 111-118 (2003).

Wawer, M. J. et al. Rates of HIV-1 transmission per coital act, by stage of HIV-1 infection, in Rakai, Uganda. J. Infect. Dis. 191, 1403-1409 (2005).

Lovejoy, T. E. & Hannah, O., eds. Climate Change and Biodiversity, 418 pp. (Yale University Press, New Haven, 2005).

Metzgar, D., Myers, C. A., Russell, K. L., Faix, D., Blair, P. J., et al. Single Assay for Simultaneous Detection and Differential Identification of Human and Avian Influenza Virus Types, Subtypes, and Emergent Variants. PLoS ONE 5(2): e8995. Doi:10.1371/journal.pone.0008995 (2010).

What is claimed is:

1. A method for monitoring the physical condition or health status of a traveler comprising:
a) selecting at least one traveler for an evaluation of said traveler's physical condition or health status;
b) obtaining at least one specimen from said traveler;
c) subjecting said specimen to at least one test that is diagnostic for at least one parameter of the physical condition or health status of the traveler, said test conducted prior to or while the traveler undertakes a journey; and
d) reporting results of said diagnostic test prior to or contemporaneous with the arrival of the traveler at the traveler's destination.

2. The method of claim 1 further comprising:
e) communicating results of the diagnostic test to the traveler's destination prior to or contemporaneous with the arrival of the traveler; and
f) determining the status of and/or further activities involving the traveler based upon the test results contained in said report.

3. The method of claim 2 wherein said further activities comprise treating said traveler for any physical or health concerns identified in said results.

4. The method of claim 2 wherein said further activities comprise isolating said traveler for such times and conditions as are dictated by any physical or health concerns identified in said results.

5. The method of claim 2 wherein said further activities comprise refusing to permit further travel within said destination by said traveler as a consequence of any physical or health concerns identified in said results.

6. A method for monitoring the physical condition or health status of a traveler comprising:
a) selecting at least one traveler for an evaluation of said traveler's physical condition or health status;
b) obtaining at least one specimen from said traveler;
c) subjecting said specimen to at least one test that identifies the presence of an infectious agent, said test conducted prior to or while the traveler undertakes a journey; and
d) reporting results of said test prior to or contemporaneous with the arrival of the traveler at the traveler's destination.

7. The method of claim 6, further comprising:
e) communicating the results to the traveler's destination; and
f) determining the status of and/or further activities involving the traveler based upon the test results contained in said report.

8. A preconfigured unit that can be deployed in a timely way in proximity to or within a travel embarkation setting, said unit comprising sample collection materials and sample storage to facilitate rapid sample testing, to allow practice of a method for verifying the physical condition or health status of a traveler obtaining the results of at least one test that identifies the presence of at least one infectious agent in a traveler previously selected for an evaluation.

9. The preconfigured unit of claim 8, further comprising at least two of the following:
consent forms; signage; blood collection equipment; packaging for transporting a sample;
electronic communication equipment; bar code or 2D code printer or reader; boarding pass or passport scanner; sample labeling equipment; thermal human scanning equipment; human temperature measurement equipment; digital photography or video equipment; and sample testing equipment to perform a diagnostic assay on a sample taken from a traveler.

10. The preconfigured unit of claim 8, wherein the preconfigured unit allows practice of a method for monitoring the physical condition or health status of a traveler for at least one parameter of the physical condition or health status of a traveler according to claim 1.

11. The preconfigured unit of claim 8, wherein the preconfigured unit allows practice of a method for monitoring the physical condition or health status of a traveler for at least one parameter of the physical condition or health status of a traveler according to claim 6.
